(19) United States
(12) Patent Application Publication
(10) Pub. No.: US 2025/0186104 A1
(43) Pub. Date: Jun. 12, 2025

(54) MEDICAL DEVICES FOR TISSUE REMOVAL AND RELATED METHODS

(71) Applicant: Boston Scientific Scimed Inc., Maple Grove, MN (US)

(72) Inventors: Emma HORGAN, Galway (IE); Enda CONNAUGHTON, Co. Galway (IE); Aoife O’ROURKE, Co. Galway (IE); Jack O’MEARA, Ballinasloe (IE); Orlaith McELVANEY, Riverside (IE)

(73) Assignee: Boston Scientific Scimed Inc., Maple Grove, MN (US)

(21) Appl. No.: 18/970,041

(22) Filed: Dec. 5, 2024

Related U.S. Application Data
(60) Provisional application No. 63/606,683, filed on Dec. 6, 2023.

(51) Int. Cl. A61B 18/14; A61B 18/00

(52) U.S. Cl. CPC A61B 18/1445; A61B 2018/00267; A61B 2018/00601; A61B 2018/00982; A61B 2018/146

(57) ABSTRACT

Medical devices for tissue removal and related methods are described. The medical device includes a handle, including a stationary portion and a movable portion. The medical device includes a shaft. The shaft includes a sheath and one or more wires. The sheath is coupled to the stationary portion of the handle. The one or more wires are coupled to the movable portion of the handle. The medical device includes an end effector positioned at a distal end of the shaft. The end effector includes a basket. The basket includes a rim extending along an opening of the basket. The end effector includes a paddle. The end effector includes a hinge. The basket and paddle are pivotally connected to the hinge.

### CROSS-REFERENCE TO RELATED APPLICATIONS

This application claims the benefit of priority of U.S. Provisional Patent Application No. 63/606,683, filed Dec. 6, 2023, the entirety of which is incorporated herein by reference.

### TECHNICAL FIELD

Various aspects of this disclosure generally relate to medical devices for tissue removal and related methods. In particular, aspects of this disclosure relate to medical devices for, for example, manipulating, resecting, grasping, and/or collecting tissue, such as, for example, capture devices.

### BACKGROUND

Medical devices, such as endoscopes or other suitable introduction devices, are employed for a variety of diagnostic and surgical procedures, such as endoscopy, laparoscopy, arthroscopy, gynoscopy, thoracoscopy, and cystoscopy, etc. Many of these procedures are carried out for purposes of tissue resection, which may include removal of tissue of an organ or a gland to treat tumors, infestations, and the like. In particular, such procedures may be carried out by inserting an insertion device into a patient's body through a surgical incision, or via a natural anatomical orifice (e.g., mouth, vagina, or rectum), and performing the procedure or operation.

Snare devices, in particular, have been used in many medical procedures, including Endoscopic Mucosal Resection (EMR) and Endoscopic Sub-mucosal Resection (ESR), polypectomy, mucosectomy, etc., for resecting tissue from a target site. A snare device may include a snare loop formed by snare wire(s), and the snare loop engages the tissue intended to be resected. The snare loop is controlled and operated at a proximal end of the device through a suitable actuating mechanism.

However, in many conventional snare devices, the tissue may eject, disconnect, or otherwise escape from the snare once severed from the target site. Not only should the tissue be recaptured, but the tissue may also move out of the medical professional's field of view, further complicating recapture and/or resection.

The medical devices and methods of the current disclosure may rectify some of the deficiencies described above or address other aspects of the art.

### SUMMARY

Each of the aspects disclosed herein may include one or more of the features described in connection with any of the other disclosed aspects.

The disclosure includes medical devices for tissue removal and related methods useful for removing tissue, including polyps within the gastrointestinal system. For example, the disclosure includes a medical device. The medical device may include a handle. The handle may include a stationary portion and a movable portion. The medical device may include a shaft. The shaft may include a sheath and one or more wires. The sheath may be coupled to the stationary portion of the handle. The one or more wires may be coupled to the moveable portion of the handle. The medical device may include an end effector positioned at a distal end of the shaft. The end effector may include a basket. The basket may include a rim extending along an opening of the basket. The end effector may include a paddle. The end effector may include a hinge. The basket and paddle may be pivotally connected to the hinge.

The medical device may include one or more of the following aspects. The basket may be formed from a basket mesh. The paddle may be formed from a paddle mesh. The basket mesh of the basket and the paddle mesh of the paddle may be formed from a non-conductive material. The paddle mesh of the paddle may be more rigid than the basket mesh of the basket.

The rim may be formed from a conductive material. The rim may be configured to be energized with RF energy and may be configured to cut through tissue.

The movable portion of the handle may include an actuator operably connected to the end effector via the one or more wires. Proximal movement of the actuator may be configured to transition the end effector from a closed configuration to an open configuration. The medical device may include a biasing element. The biasing element may be positioned in the handle and proximal to the actuator. The biasing element may be configured to bias the actuator distally to transition the end effector from the open configuration to the closed configuration and maintain the end effector in the closed configuration. The biasing element may be a spring. During a transition from the closed configuration to the open configuration and a transition from the open configuration and the closed configuration, only one of the basket and paddle may pivot about the hinge while the other of the basket and the paddle may remain stationary. During a transition from the closed configuration to the open configuration and a transition from the open configuration and the closed configuration, both the basket and the paddle may pivot about the hinge.

The basket may include a hemi-ellipsoid shape. The paddle may be sized and shaped to cover the opening of the basket. The opening may include an elliptical shape.

A proximal end of the paddle and a proximal end of the basket may be pivotally connected to the hinge.

The disclosure may also include a method of using a medical system. The medical system may include an insertion device. The insertion device may include a working channel and a medical device receivable within the working channel. The medical device may include an end effector positioned at a distal end of a shaft of the medical device. The end effector may include a basket. The basket may include a rim extending along an opening of the basket. The rim may be configured to be energized with RF and may be configured to cut through tissue when energized. The end effector may include a paddle and a hinge. The basket and the paddle may be pivotally connected to the hinge. The medical device may include an actuator configured to transition the end effector from a closed position to an open position. The end effector may include a biasing element configured to transition the end effector from the open position to the closed position. The method may include delivering the medical device to a treatment site. The method may also include positioning the medical device over a target. The method may further include energizing the medical device to at least partially sever the target. The method may also include capturing the target. The method may still further include removing the target from the treatment site.

The method may include one or more of the following aspects. During the extending step, one of the basket and the paddle may be positioned about one side of the target, and the other of the basket and the paddle may be positioned about another opposite side of the target. The method may include, after the capturing step, de-energizing the rim after receiving the target within the basket. The method may include, during the capturing step, visualizing the capturing of the target via a camera of the medical system.

The disclosure may include a medical system. The medical system may include a handle. The handle may include an actuator and a biasing element. The medical system may include a shaft. The shaft may include at least one wire. A proximal end of the wire may be coupled to the actuator. The medical system may include a medical device. The medical device may include an end effector. The end effector may be positioned at a distal end of the shaft and may be coupled to a distal end of the wire. The end effector may include a basket. The basket may include a rim extending along an opening of the basket. The end effector may include a paddle. The end effector may include a hinge. The basket and the paddle may be pivotally connected to the hinge at a proximal end of the basket and a proximal end of the paddle. Movement of the actuator from a first position to a second position on the handle may transition the end effector from a closed configuration to an open configuration. The biasing element may urge the actuator toward the first position on the handle to transition the end effector from the open configuration to the closed configuration.

### BRIEF DESCRIPTION OF THE FIGURES

The accompanying drawings, which are incorporated in and constitute a part of this specification, illustrate various aspects of this disclosure and together with the description, serve to explain the principles of the disclosure.

FIG. 1 depicts an exemplary medical system.

FIG. 2A depicts a top view of a distal end of the medical device in an open configuration near a target site.

FIG. 2B depicts a side view of the distal end in the open configuration near the target site.

FIG. 3A depicts a top view of the distal end in the open configuration surrounding the target site.

FIG. 3B depicts a side view of the distal end in the open configuration surrounding the target site.

FIG. 4A depicts a top view of the distal end in a closed configuration.

FIG. 4B depicts a side view of the distal end in the closed configuration.

FIG. 5 depicts a flow chart of a method using the exemplary medical system to capture and retain tissue.

### DETAILED DESCRIPTION OF THE FIGURES

Particular aspects of the disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of +10% in a stated numeric value or range.

This disclosure is drawn to medical devices such as tissue removal devices, and related methods of use thereof.

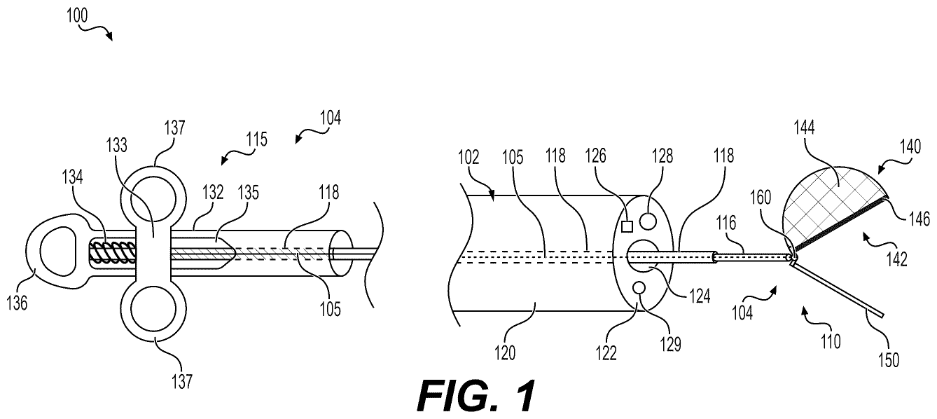

FIG. 1 depicts various aspects of an exemplary medical system 100 including an insertion device 102 and a medical device 104. Insertion device 102 includes a shaft 120, and, in some aspects, insertion device 102 may include a handle (not shown). Shaft 120 includes one or more working channels 124. Medical device 104 may be delivered to a treatment site via insertion device 102, for example, through working channel 124. Additionally, medical device 104 includes an end effector 110 that may be manipulated to transition between a closed configuration and an open configuration. Furthermore, medical device 104 may include one or more conductive portions, for example, to help cut tissue. In these aspects, and as discussed in detail below, medical device 104 may be extended (e.g., from insertion device 102) and manipulated to cut and capture a target (e.g., a polyp or other tissue).

The handle of insertion device 102 may be positioned at a proximal end of shaft 120. Shaft 120 may be generally tubular, and may extend from a distal end of a handle. Shaft 120 includes a distal end 122. One or more working channels 124 may extend at least from a proximal end of shaft 120 (e.g., that is adjacent to the distal end of the handle) to distal end 122. Shaft 120 may define multiple working channels, one or more fluid channels, and/or one or more lumens. Working channel 124 may be sized and shaped to receive at least a portion of end effector 110. Distal end 122 may include an illumination device or light 126 and/or may include a visualization device or camera 128. Distal end 122 may include one or more fluid channels 129. Fluid channel(s) 129 may be a fluid delivery channel and/or may be a suction or negative pressure channel. The handle of insertion device 102 may include one or more ports and one or more valves.

End effector 110 may include a basket 140 and a paddle 150. End effector 110 may include a hinge 160. Hinge 160 may connect basket 140 and paddle 150, for example, proximal ends of basket 140 and paddle 150. Basket 140 and paddle 150 may each pivot about hinge 160 in a scissor-like motion. Basket 140 and paddle 150 may pivot about the hinge 160 in tandem or independently/separately of one another. Alternatively, end effector 110 may include a scissor/scissor-like mechanism (not shown) to close and open end effector 110. End effector 110 may be actuated via a one or more wires 105 disposed within a lumen of a medical device shaft 116. In some embodiments, basket 140 may be actuated by one wire of the one or more wires 105 disposed within the lumen of shaft 116, and paddle 150 may be actuated by another wire of the one or more wires 105 disposed within the lumen of shaft 116. In other embodiments, basket 140 or paddle 150 may be actuated by one of the one or more wires 105, while the other of the basket 140 or paddle 150 may remain stationary. In some embodiments, basket 140 and paddle 150 may both be actuated by a single wire 105. End effector 110 may be positioned at a distal end of shaft 116. At least a portion of shaft 116 may be received by a sheath 118. At least a portion of each of sheath 118, shaft 116, one or more wires 105, and end effector 110 may be received within working channel 124. At least a portion of sheath 118 may be received within working channel 124. End effector 110 may be sized and shaped to capture a target 130 positioned within a body of a patient.

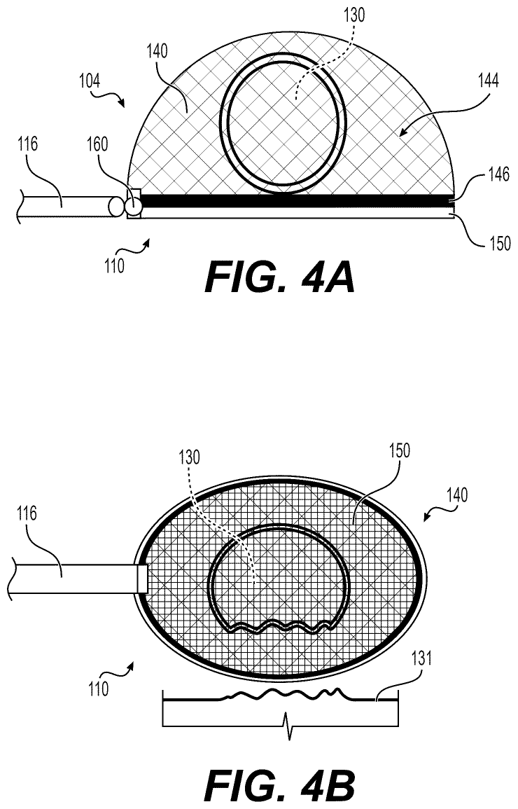

Additionally, in some aspects, medical device 104 includes a handle 115, including a stationary portion (e.g. a main body) 132 and a movable portion or actuator 133. One or more wires 105 may be actuated by actuator 133. In embodiments with multiple wires 105, there may be one or more actuators 133. Wire 105 may be attached or otherwise operably connected to actuator 133. Moving actuator 133 may move wire 105, thereby actuating one or more components of end effector 110 (e.g. basket 140 and/or paddle 150). Actuator 133 may be engaged with a slot 135 of handle 115, for example, of stationary portion 132, and may slide along slot 135. Handle 115 may include a biasing element 134. Biasing element 134 may be positioned between a distal facing surface of stationary portion 132 and a proximal facing surface of actuator 133. Pulling actuator 133 proximally may pull wire 105, thereby actuating end effector 110 and compress biasing element 134. Upon releasing actuator 133, actuator 133 of handle 115 may be returned to an original position by biasing element 134. Alternatively, or in addition to biasing element 134, actuator 133 may be returned to the original position by the user. Actuating one or more wires 105 via actuator 133 may transition the end effector 110 from a closed position (at least shown in FIG. 4A) to an open position (at least shown in FIG. 1). Ceasing actuation of one or more wires 105 may transition the end effector 110 from the open position to the closed position. Handle 115, for example, stationary portion 132, may include a ring 136 (e.g., a thumb ring) at a proximal end of handle 115, for example, stationary portion 132. Actuator 133 may include one or more rings 137. Rings 136, 137 may be configured to receive a finger or digit.

Basket 140 may be sized and/or shaped to receive target 130. Basket 140 may be hemi-ellipsoid. However, basket 140 is not limited to hemi-ellipsoidal shapes, and may be any shape (such as a hemi-spherical shape or hemi-prismatic shape). Basket 140 may be formed from a mesh 144 for example, a basket mesh. Mesh 144 may be formed from Nitinol (a nickel titanium alloy), other similar alloys used within the medical industry, a suture material (e.g., nylon, polypropylene, silk, or polyester to weave or knit the mesh), silicone or plastic, other similar materials, and combinations thereof. Mesh 144 may be configured to be at least partially collapsible and expandable, so that basket 140 may be at least partially collapsed and received within an working channel 124 and/or sheath 118 and may expand so that basket 140 is at least capable of receiving target 130 after exiting the distal opening of shaft 116.

Basket 140 includes an opening 142. Opening 142 may be sized and shaped to receive target 130. Opening 142 may be positioned at a widest portion of the basket 140, such as but not limited to a base of the hemi-ellipsoid. Opening 142 may include an ellipse or elliptical shape. Basket 140 may include a rim 146. Rim 146 may extend along a portion of or an entirety of a periphery of opening 142. In other words, rim 146 may extend along a periphery of opening 142. Rim 146 and/or the periphery of opening 142 may be shaped as the base of basket 140. Rim 146 may be formed from a conductive material, and mesh 144 may be formed from a non-conductive material. Rim 146 may conduct radio frequency (“RF”) energy, electrical current, heat ablation, cold ablation, microwave ablation, ultrasound energy, and other energy types used by those skilled in the art. Rim 146 may be more rigid than mesh 144. A proximal end of basket 140 may be pivotally connected a hinge 160. The proximal end of basket 140 may pivot about hinge 160.

In some embodiments, both rim 146 and mesh 144 may be formed from a conductive material. In these aspects, both rim 146 and mesh 144 may conduct RF energy, electrical current, heat ablation, cold ablation, microwave ablation, ultrasound energy, and other energy types used by those skilled in the art.

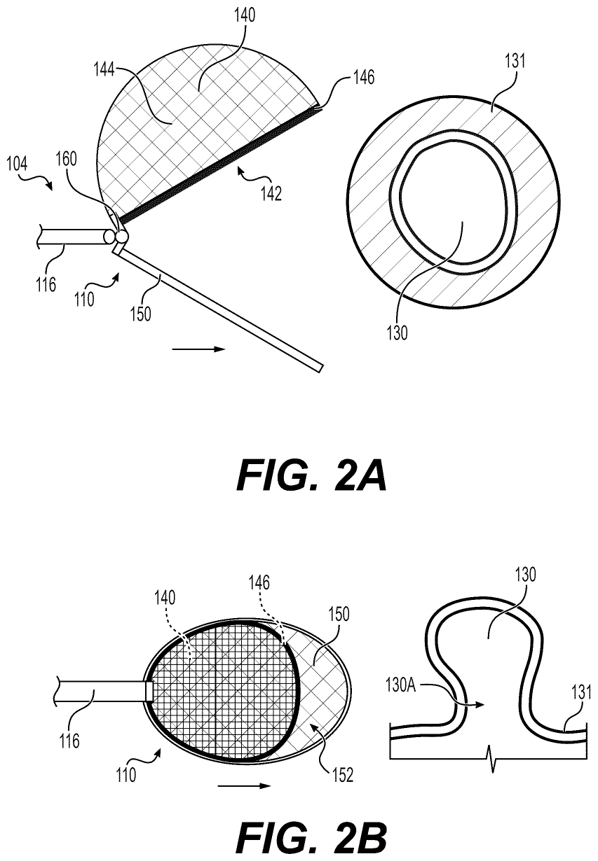

Paddle 150 may be sized and/or shaped to at least partially cover opening 142 of basket 140. Paddle 150 may be the same shape and/or size as opening 142. In some aspects, paddle 150 may be slightly smaller than opening 142. In other aspects, paddle 150 may be slightly larger than opening 142. A proximal end of paddle 150 may be pivotally connected to hinge 160, for example, allowing basket 140 and/or paddle 150 to transition between a first configuration, in which opening 142 is at least partially exposed, and a second configuration, in which opening 142 is at least partially closed by paddle 150. Paddle 150 may be formed from a mesh 152 (as at least seen in FIG. 2A), for example, a paddle mesh. Mesh 152 may be equally or more rigid than mesh 144. At least a portion of mesh 152 may be formed from a non-conductive or insulative material. Mesh 152 may be formed from Nitinol (a nickel titanium alloy), other similar alloys used within the medical industry, a suture material (e.g., nylon, polypropylene, silk, polyester to weave or knit the mesh), silicone or plastic, other similar materials, and combinations thereof. Mesh 144 and mesh 152 may be configured to conform, change in size and/or shape, or contort when contacting dimensions of the target 130, for example, to receive and/or accommodate target 130 and/or help to prevent target 130 from escaping out of opening 142 of basket 140.

In alternate embodiments, one or more of basket 140 and paddle 150 may be formed from different material instead of meshes 144, 152, respectively. Basket 140 and/or paddle 150 may be formed from a compressible and/or expandable material. In some aspects, the material of paddle 150 may be equally or more rigid than the material of basket 140.

FIGS. 2A-4B illustrate an exemplary procedure for capturing and removing target 130 with medical device 104. FIGS. 2A, 3A, and 4A depict a downward facing view of target 130 and end effector 110 (i.e., a top-down view), such that a top portion of target 130 is shown (as opposed to a bottom portion of target 130 which would be connected to living tissue, viewable in FIGS. 2B, 3B, and 4B). FIGS. 2B, 3B, and 4B depict a side view of target 130 and end effector 110. In these aspects, FIGS. 2B, 3B, and 4B show target 130 extending from other surrounding tissue at a treatment site 131 (e.g., an interior surface of a colon).

FIGS. 2A and 2B depict end effector 110 approaching target 130 (e.g., a polyp), with end effector 110 in an open configuration. Although end effector 110 is shown in the open configuration in FIGS. 2A and 2B, it should be understood that end effector 110 may initially be in a closed configuration at the beginning of the procedure, similar to the closed configuration shown in FIG. 4A without target 130 contained within end effector 110. It should be further understood that end effector 110 may be biased toward the closed configuration by biasing element 134. In this aspect, after actuator 133 is actuated and transitions end effector 110 from the closed configuration to the open configuration, deactuating actuator 133 allows biasing element 134 to transition end effector 110 from the open configuration to the closed configuration. Actuating actuator 133 may include applying a force to actuator 133 in a proximal direction. Deactuating actuator 133 may include releasing the force applied to actuator 133 in the proximal direction, which may allow biasing element 134 to apply a force to actuator 133 in a distal direction to move actuator distally. The distal movement of actuator 133 may then transition end effector 110 from the open configuration to the closed configuration. Alternatively or additionally, deactuating actuator 133 may include the user applying a force to actuator in the distal direction to move actuator 133, and corresponding transition end effector 110. The closed configuration may be the default state of end effector 110 when distal to an opening of working channel 124. Additionally, biasing element 134 may help to maintain end effector 110 in the closed configuration absent actuator 133 being actuated again.

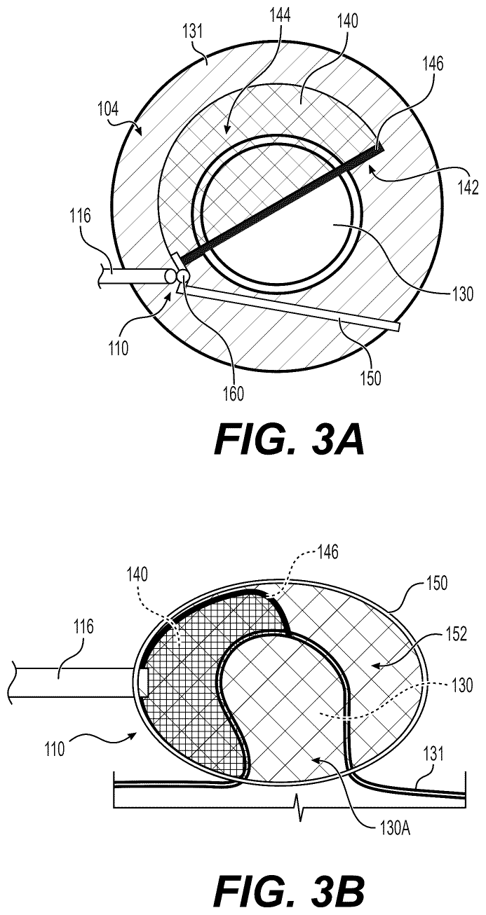

In the open configuration, end effector 110 may be configured to receive target 130. With end effector 110 in the open configuration, end effector 110 may be advanced in the distal direction until end effector 110 is positioned so that at least a portion of target 130 is positioned between basket 140 and paddle 150. In some aspects, end effector 110 may be advanced until end effector 110 is positioned around target 130, similarly to FIGS. 3A and 3B. In these aspects, target 130 may be receivable within basket 140 once at least the portion of target 130 is severed from treatment site 131.

FIGS. 3A and 3B depict end effector 110 positioned around target 130, so that target 130 is positioned between basket 140 and paddle 150. Additionally, as mentioned above, target 130 may be receivable within basket 140 once at least the portion of target 130 is severed from treatment site 131. Rim 146 may be energized, for example, with RF energy, once target 130 is positioned between basket 140 and paddle 150. Alternatively or additionally, rim 146 may be energized before target 130 is positioned between basket 140 and paddle 150. Basket 140 may be positioned on one side of target 130, and paddle 150 may be positioned on another side of target 130 opposite to basket 140. In these aspects, when end effector 110 is transitioned into a closed positioned, energized rim 146 may help to cut and/or burn through a bottom portion 130A of target 130 (e.g., such as a stalk of a polyp) while paddle 150 applies force against target 130 in the direction of basket 140 and/or energized rim 146 to help facilitate the cutting and/or burning through of bottom portion 130A of target 130. For example, after target 130 has been positioned between basket 140 and paddle 150, end effector 110 may be transitioned from the open configuration to the closed configuration. End effector 110 may be transitioned from the open configuration to the closed configuration via biasing element 134 and/or the user moving actuator 133. As end effector 110 is transitioned from the open position to the closed position, rim 146 may cut through bottom portion 130A of target 130, severing target 130 from treatment site 131. Additionally, basket 140 may capture or otherwise retain the severed target 130 within basket 140, for example, with paddle 150 at least partially covering opening 142 of basket 140. In these aspects, basket 140 and paddle 150 may help to prevent target 130 from exiting basket 140. After being transitioned into the closed configuration with target 130 contained with basket 140, end effector 110 and target 130 may resemble the example shown in FIGS. 4A and 4B.

FIGS. 4A and 4B depict end effector 110 in a closed position with severed target 130 contain within basket 140. Additionally, paddle 150 may at least partially cover and/or seal opening 142, preventing target 130 from exiting opening 142 until desired by the user. After target 130 has been severed from treatment site 131 via energized rim 146, rim 146 may be de-energized. End effector 110 with target 130 contained therein may be retracted proximally toward working channel 124 and shaft 120. After capturing target 130 with end effector 110, shaft 120 and end effector 110 may be removed from the patient, thus also removing target 130. Alternatively, after capturing target 130, if target 130 is small enough to pass through fluid channel 129, target 130 may be released from end effector 110 and suction may be applied via fluid channel 129 to retrieve target 130 and allow for end effector 110 to be used to capture another, different target 130.

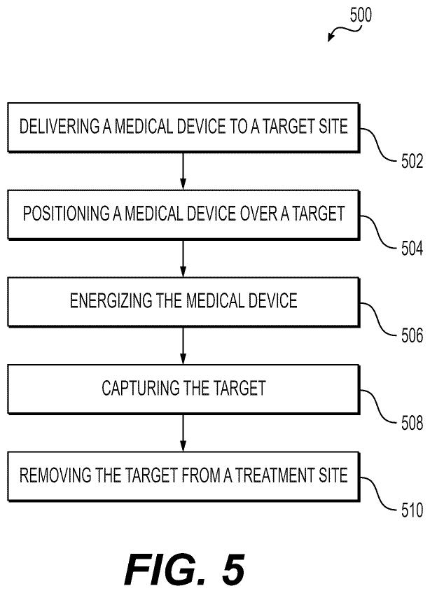

As seen in FIG. 5, this disclosure includes a method 500 for tissue removal using medical system 100. Method 500 includes the initial step 502 of delivering the medical to a treatment site. Initial step 502 may include inserting insertion device 102 into a bodily lumen or orifice (natural or man-made incision). Initial step 502 may also include an initial step of positioning distal end 122 of shaft 120 near a target (e.g., target 130), distal end 122 may be facing the target, for example, with target positioned adjacent to or distal of distal end 122. Initial step 502 may include the initial step of advancing medical device 104 (e.g., end effector 110) in the direction of the target, as seen at least in FIGS. 2A and 2B and indicated by the directional arrows in FIGS. 2A and 2B.

Initial step 502 may include the initial step of transitioning end effector 110 from the closed position to the open position, for example, via movement of one or more portions of handle 115. End effector 110 may be transitioned from the closed configuration to the open configuration by actuating or deactuating actuator 133. In the open position, end effector 110 may be capable of receiving the target and capable of capturing the target after been biopsied (or otherwise cut from) from the treatment site 131. End effector 110 may be transitioned from the closed position to the open position after end effector 110 has advanced sufficiently away from distal end 122 and/or an opening of working channel 124, so that end effector 110 may expand from a collapsed configuration (not shown) to an expanded configuration (shown in FIGS. 1-4B).

In the collapsed configuration, end effector 110 may be distorted or reshaped into a shape configured to be received within working channel 124. The material, such as but not limited to Nitinol, forming end effector 110 (e.g., basket 140 and/or paddle 150) may help end effector 110 transition from the collapsed configuration to the expanded configuration after end effector 110 as exited the opening of working channel 124. In some embodiments, working channel 124 may be of sufficient size so that end effector 110 may be in an at least partially expanded configuration within working channel 124. In alternate embodiments, end effector 110 may be sufficiently small to be received within working channel 124 in an expanded configuration.

Method 500 includes a step 504 of positioning the medical device over the target. For example, medical device 104 may be positioned over target 130. In these aspects, end effector 110, in the open configuration, may be positioned so that basket 140 is positioned on a first side of target 130 and paddle 150 is positioned on second side of target 130, opposite to the first side (as seen in FIGS. 3A and 3B). Basket 140 may be positioned so that when end effector 110 is transitioned from the open configuration to the closed configuration, target 130 is received within basket 140.

Method 500 includes a step 506 of energizing the medical device. For example, rim 146 of medical device 104 may be energized with RF energy or other appropriate type of energy, as described in preceding paragraphs of this disclosure. Insertion device 102 and/or medical system 100 may include appropriate conductors, circuitry, energy sources, and/or energy to energize rim 146. Rim 146 may be energized such that it may cut through and/or burn through a portion of target 130 and/or the treatment site 131 (e.g., such as a stalk of a polyp) so as to cut target 130 from the treatment site 131.

Method 500 includes a step 508 of capturing the target. For example, end effector 110 may be transitioned from the open configuration to the closed configuration while end effector 110 at least partially surrounds target 130, receiving target 130. End effector 110 may be transitioned from the open configuration to the closed configuration by actuating or deactuating actuator 133. As end effector 110 is transitioned from the open configuration to the closed configuration, energized rim 146 may contact bottom portion 130A of target 130 and/or treatment site 131and cut through and/or burn through bottom portion 130A of target 130 and/or treatment site 131. After end effector 110 has transitioned from the open configuration to the closed configuration, energized rim 146 may have cut through the entirety of bottom portion 130A of the target and/or treatment site, severing the target from the treatment site. The severed target 130 may be received within opening 142 of basket 140 and contained within a volume of basket 140. Additionally, paddle 150 may help to prevent the severed target 130 from exiting basket 140 by at least partially covering opening 142 (as seen in FIGS. 4A and 4B). In some embodiments, end effector 110 may be transitioned from an open configuration to an at least partially closed configuration where a surface of paddle 150 presses against target 130, preventing target 130 from fully exiting the volume of basket 140. In the partially closed configuration, a dimension between a distalmost point of basket 140 and a distalmost point of paddle 150 may be too small to allow target 130 to fully exit the volume of basket 140. After capturing target 130, step 508 may include de-energizing or otherwise termination the energy delivery to end effector 110 (e.g., rim 146). Step 508 may further include visualizing the capturing of target 130 via the elements on the distal end (e.g. camera 128) of the insertion device 102.

Method 500 includes a step 510 of removing the target from the treatment site. For example, after the severed target 130 has been captured by end effector 110, insertion device 102 may be removed from the bodily lumen or orifice. After severed target has been capture by end effector 110, end effector 110 may be retracted proximally, in a direction toward distal end 122 of shaft 120. In embodiments where working channel 124 is sufficient large to receive end effector 110 in the expanded configuration, end effector 110 and target 130 may be at least partially received within working channel 124. For example, end effector 110 and target 130 may be removed, while shaft 120 of insertion device 102 remains at the treatment site.

In some aspects, another medical device with another end effector may be delivered through insertion device 102 to treat the treatment site. Alternatively, medical device 104 may be re-delivered through insertion device 102 to further treat the treatment site, for example, after target 130 has been removed from end effector 110. Further, in embodiments where end effector 110 is sufficiently small to be received within working channel 124 in the expanded configuration, end effector 110 and target 130 may be at least partially received within working channel 124. In some aspects, after end effector 110 and target 130 have been retracted proximally toward distal end 122 of shaft 120, insertion device 102 may be removed from the bodily lumen or orifice, thus removing the entirety of medical system 100 and target 130 from the treatment site. Step 510 may include removing some or all components of the medical system 100 of the body of the patient. Step 510 may include removing medical device 104 with target 130 retained within end effector 110 through working channel 124 of insertion device 102.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical device comprising:
a handle, including a stationary portion and a movable portion;
a shaft, including a sheath and one or more wires, wherein the sheath is coupled to the stationary portion of the handle, and wherein the one or more wires are coupled to the movable portion of the handle; and
an end effector positioned at a distal end of the shaft; and the end effector comprising:
a basket, the basket including a rim extending along an opening of the basket,
a paddle, and
a hinge, wherein the basket and the paddle are pivotally connected to the hinge.

2. The medical device of claim 1, wherein the basket is formed from a basket mesh, and wherein the paddle is formed from a paddle mesh.

3. The medical device of claim 2, wherein the basket mesh of the basket and the paddle mesh of the paddle are both formed from a non-conductive material.

4. The medical device of claim 2, wherein the paddle mesh of the paddle is more rigid than the basket mesh of the basket.

5. The medical device of claim 1, wherein the rim is formed from a conductive material.

6. The medical device of claim 5, wherein the rim is configured to be energized with RF energy and is configured to cut through tissue.

7. The medical device of claim 1, wherein the movable portion of the handle includes an actuator operably connected to the end effector via the one or more wires, wherein proximal movement of the actuator is configured to transition the end effector from a closed configuration to an open configuration.

8. The medical device of claim 7, further comprising a biasing element, wherein the biasing element is positioned in the handle and proximal to the actuator, wherein the biasing element is configured to bias the actuator distally to transition the end effector from the open configuration to the closed configuration and maintain the end effector in the closed configuration.

9. The medical device of claim 8, wherein the biasing element is a spring.

10. The medical device of claim 7, wherein during a transition from the closed configuration to the open configuration and a transition from the open configuration and the closed configuration, only one of the basket and the paddle pivots about the hinge while the other of the basket and the paddle remains stationary.

11. The medical device of claim 7, wherein during a transition from the closed configuration to the open configuration and a transition from the open configuration and the closed configuration, both the basket and the paddle are configured to pivot about the hinge.

12. The medical device of claim 1, wherein the basket includes a hemi-ellipsoid shape.

13. The medical device of claim 12, wherein the paddle is sized and shaped to cover the opening of the basket.

14. The medical device of claim 13, wherein the opening includes an elliptical shape.

15. The medical device of claim 1, wherein a proximal end of the paddle and a proximal end of the basket are pivotally connected to the hinge.

16. A method of using a medical system, wherein the medical system comprising: an insertion device including a working channel and an medical device receivable within the working channel, the medical device including an end effector positioned at a distal end of a shaft of the medical device; the end effector comprising: a basket, the basket including a rim extending along an opening of the basket, the rim configured to be energized with RF and configured to cut through tissue when energized, a paddle, a hinge, wherein the basket and the paddle are pivotally connected to the hinge, an actuator configured to transition the end effector from a closed position to an open position, and a biasing element configured to transition the end effector from the open position to the closed position; the method comprising:
delivering the medical device to a treatment site;
positioning the medical device of over a target;
energizing the medical device to at least partially sever the target;
capturing the target at least partially within the basket; and
removing the target from the treatment site.

17. The method of claim 16, wherein during the positioning step, one of the basket and the paddle is positioned about one side of the target and the other of the basket and the paddle is position about another opposite side of the target.

18. The method of claim 16, further comprising: after the capturing step, de-energizing the rim after receiving the target within the basket.

19. The method of claim 18, further comprising: during the capturing step, visualizing the capturing of the target via a camera of the medical system.

20. A medical system comprising:
A handle, including an actuator and a biasing element;
a shaft, including at least one wire, wherein a proximal end of the at least one wire is coupled to the actuator; and
a medical device including an end effector, the end effector positioned at a distal end of the shaft and coupled to a distal end of the at least one wire, the end effector comprising:
a basket, the basket including a rim extending along an opening of the basket,
a paddle, and
a hinge, wherein the basket and the paddle are pivotally connected to the hinge at a proximal end of the basket and a proximal end of the paddle;
wherein movement of the actuator from a first position to a second position on the handle transitions the end effector from a closed configuration to an open configuration, and wherein the biasing element urges the actuator toward the first position on the handle to transition the end effector from the open configuration to the closed configuration.
